(19) United States
(12) Patent Application Publication
(10) Pub. No.: US 2022/0315974 A1
(43) Pub. Date: Oct. 6, 2022

(54) INDOOR BIOLOGICAL DETECTION SYSTEM AND METHOD

(71) Applicant: ENSCO, Inc., Springfield, VA (US)

(72) Inventors: Kevin Michael DENELSBECK, Melbourne, FL (US); Gaylen Wayne DRAPÉ, Melbourne, FL (US); Matthew William MCGARVEY, Springfield, VA (US); Shawn ROTTMANN, Melbourne, FL (US)

(21) Appl. No.: 17/220,554

(22) Filed: Apr. 1, 2021

(51) Int. Cl. C12Q 1/02; C12Q 1/686

(52) U.S. Cl. CPC C12Q 1/02; C12Q 1/686

(57) ABSTRACT

A method for indoor biological detection of a monitored space is provided including collecting and entering monitored space information to determine density and location of sensors for monitoring air for an aerosol plume, distributing the sensors throughout the space, monitoring air and detecting and characterizing a plume event, determining a source location, collecting and preparing an air sample upon the detection of the plume event, and assaying the air sample to identify a hazardous release utilizing a field screening device. The method continues with the steps of initiating a precautionary response for the hazardous release characterizing the plume as biological or non-biological and initiating a protective response. A system is also provided.

### BACKGROUND OF THE INVENTION

The present invention is directed to detection of biological agents in indoor spaces. More particularly, the present invention is directed to a system and method for detection and response to introduction of biological agents to protect the public in indoor spaces.

Prior art operational biological surveillance systems provide information hours or days after an intentional attack or accidental introduction of aerosolized biological agents, requiring manual additional sampling and information gathering activities to understand if the event was a public health concern.

Prior art rapid biological detection equipment is expensive and non-automated. Deployment of a larger number of sensors using the prior art would be needed to achieve the required detection performance but would cause the detection architecture to be cost prohibitive for a system which is dedicated to solely monitoring for a low probability event.

Prior systems are typically comprised of loosely coupled, independent arrays of biosensors and detectors. Particulate and bioaerosol sensors increase awareness that potentially harmful bioagents are present in the building. Air collectors and biological identification devices verify presence of dangerous bioagents. Biosensors, air collectors, and identifiers are typically government developed or heavily subsidized by government agencies, functionally sophisticated, and therefore extremely expensive with a purchase price of twenty thousand to hundreds of thousand dollars each.

In these prior systems, data streams from these multiple sensors are monitored individually by human operators through a common operational workstation. Software displays data from individual sensors and generates alarms based on simple rules. For example, an alarm sounds when a monitored concentration from a sensor at time X exceeds threshold Y (where Y is constant). Software collates the sensor alarms using rule-based event processing, and temporally groups alarms into “events” for each individual sensor type. An operator visually observes the type and location of events, manually checks the sensor display, issues warnings, and initiates appropriate action via a checklist. On a post-incident basis, indoor plume (physical) models are initiated manually by a subject matter expert and executed asynchronously on a separate workstation for purposes of contamination mapping, building decontamination, and forensic assessment.

There are numerous drawbacks to these prior art systems. A first drawback is that analysis of separate data streams by sensor type does not account for dynamic background or spurious events that occur in a typical building environment. This results in too many false alarms for an operator to easily respond to. A second drawback is that there is no automated integration of signals across sensor types. Operator-initiated actions from a first sensor alarm to a positive identification of a hazard are prone to human decision-making error and can result in sub-optimal responses. A third drawback of prior art systems is that the interfaces are inefficient and cannot make effective use of inexpensive biological sensors, collectors, and detectors. This results in a response time that is too slow relative to speed of hazard dispersal through the building. A fourth drawback of prior art systems is that physical modeling of hazard plumes is done after the fact. Predictive information does not play a role in the real-time incident response. Finally, a fifth drawback of the prior art is that these systems cannot accomplish a biothreat detection in a sufficiently timely manner for reasonable mitigation. That is, populations can be neither warned to evacuate nor can the detection be timely enough to provide for successful medical intervention after exposure. The current timelines for detection and confirmation of a biothreat exposure in the prior art is typically 24-48 hours.

### SUMMARY OF THE INVENTION

The present invention is directed to a system and method for rapidly detecting and responding to intentional or accidental introduction of aerosolized biological agents for purposes of protecting the public in indoor locations. The present invention is directed to a rapid biological detection system and method in an architecture that is low-cost through a multiple-tiered approach, fusion of data from multiple sensor types, and pattern recognition algorithms.

The present invention is directed to a multi-tiered, fully automated, scalable system and method that significantly improves the cost effectiveness and responsiveness of prior art systems. Some innovative aspects of this system and method include the following. First, the system and method provide early warning capability powered by real-time or pre-generated plume modeling information and intelligent pattern recognition methods. Second, the system and method enables the use of less expensive, off-the-shelf air quality monitors as trigger sensors. Third, the system and method allow for greater confidence in warnings and alarms through integrated analysis of data from multiple tiers of sensing devices and analytics. Fourth, the system and method provide for reduced cost of operation by activating biological identification detectors only when needed, i.e., when detection and characterization of hazard plume presented. Fifth, the system and method reduce false trigger warnings through correlation with modeled incident scenarios.

The system and method enable seamless integration of disparate, off-the-shelf sensing technologies and systems through scalable system architecture. The system and method include use of triggers (e.g., laser counters, fluorescence detectors); and bio-identification (e.g., air samplers, mass spectrometers, and Polymerase Chain Reaction (PCR) assays). The system and method provide rapid decision-making capability through “Operator-On-The-Loop” assistance software and analytics.

The system and method draw an operator's attention to anomalies across multivariate data streams and avoid overwhelming the operator with non-threatening patterns in individual (univariate) data streams. Finally, the system and method presumptively identify a biothreat in, for example, one hour or less, providing ample time to effect mitigation responses such as building/HVAC responses, decision-maker/first responder alerting, population warnings, and even allows time for medical intervention given exposure.

The present invention is first directed to a method for indoor biological detection of a monitored space, including the steps of collecting and entering monitored space information to determine density and location of sensors for monitoring air in the space for an aerosol plume, distributing the sensors throughout the monitored space, monitoring the air and detecting and characterizing a plume event; determining a source location and collecting and preparing an air sample upon the detection of the plume event. The method continues with the steps of assaying the air sample to identify a hazardous release, initiating a precautionary response for the hazardous release, characterizing the plume, and initiating a protective response.

The step of determining a source location may include analyzing and modeling data collected to define hazard transport and dispersion behavior to determine a contamination map. The method may include a step of mapping and monitoring dynamic plume movement within the monitored space. The step of distributing sensors may include distributing a first tier of particulate sensors and a second tier of bioaerosol sensors.

The steps of collecting and preparing an air sample and assaying the air sample may be accomplished with a presumptive identification subsystem. The steps of initiating a precautionary response for the hazardous release, characterizing the plume, mapping plume movement within the monitored space, and initiating a protective response may be accomplished with a command and control subsystem. The sensors may be, for example, particulate sensors, air collectors, and biological detectors and the like.

The method may include the steps of analyzing the transport and dispersion behavior utilizing a sensor placement algorithm to determine a number and type of the sensors and placement within the monitored space. The step of analyzing and modeling the monitored space to define the hazard transport and dispersion behavior may utilize an open source CONTAM computer program. A step of presumptively identifying material from a hazardous release may utilize a field screening device such as a Polymerase Chain Reaction (PCR) detector. The step of initiating a protective response may include, for example, changing HVAC settings, closing and opening doors, closing and opening of windows, sending e-mails, providing alerts to human operators, and providing audible alarms. The method may further include the steps of identifying where the source of the plume event started, when the plume event started and determining a best location to collect and prepare the air sample. Finally, the method may include the step of providing a video feed from a closed-circuit camera to confirm or disconfirm human activity associated with a biological agent release.

An indoor biological detection system for a monitored space is also provided which includes a trigger subsystem comprising an array of potentially different types of sensors for monitoring air for an aerosol plume, to detect and characterize a plume event; a presumptive identification subsystem comprising a system to collect and prepare an air sample upon detection of a plume event, and to assay the sample and identify threat organisms; and a command and control subsystem to initiate a precautionary response, locate the source of the plume, map plume movement within the monitored space, and initiate a protective response.

The command and control subsystem may archive data regarding the air sample. The presumptive identification subsystem may include an air collector, a sample preparation and delivery accessory, and a field screening device (e.g., a Polymerase Chain Reaction (PCR) analysis device). The trigger subsystem may include commercial-off-the-shelf sensors.

### BRIEF DESCRIPTION OF THE DRAWINGS

FIG. 1 is a simplified block diagram of the architecture of an indoor biological detection system and method in accordance with an exemplary embodiment of the present invention.

FIG. 2 is a flowchart of the functional flow of the indoor biological detection system and method of FIG. 1.

FIG. 3 is a flowchart of a Configuration Phase of the indoor biological detection system and method of FIG. 1.

FIG. 4 is a flowchart of indoor Transport and Dispersion (T&D) modeling flow, as used in the Configuration Phase of FIG. 3 of the indoor biological detection system and method of FIG. 1.

FIG. 5 is a flowchart of an Optimal Sensor Placement (OSP) forward selection algorithm as used in the Configuration Phase of FIG. 3 of the indoor biological detection system and method of FIG. 1.

FIG. 6 is a flowchart of an Operational Phase of the indoor biological detection system and method of FIG. 1.

FIG. 7 is a flowchart of the logic flow of a Plume Detector Tier 1 Event Determination of the Operational Phase of FIG. 6.

FIG. 8 is a logic flowchart of a Plume Detector Tier 2 Event Determination of the Operational Phase of FIG. 6.

FIG. 9 is a block diagram showing the relationship among the tiers of the Operational Phase of the indoor biological system and method of FIG. 6.

FIG. 10 is a flowchart for event engine high-level data flow of the Operational Phase of the indoor biological system and method of FIG. 6.

FIG. 11 is a block diagram of the physical architecture of the Operational Phase of the indoor biological system and method of FIG. 6.

### DETAILED DESCRIPTION

The indoor biological detection system and method fora monitored space such as a building or portion of a building of the present invention is a system having a flexible, scalable, and modular system architecture that easily adapts as the component state of the art evolves. The indoor biological detection system and method addresses new and emerging biological threats fora variety of indoor venues, such as office buildings and conference centers. It includes an interface with sensors for traditional and non-traditional chemical agents, toxic industrial chemicals, explosives, and radiological threats. The system functions autonomously with application across a broad spectrum of threats for complete situational awareness.

The indoor biological detection system and method of the present invention provides the following benefits:
autonomous bio-threat detection with short detection time, high probability of detection, and low false alarm rate, providing actionable information and the confidence necessary to decision makers to save lives, save money, and quickly restore normal operations;
seamless interfaces for integration with any detection system as well as its potential for use in areas such as environmental monitoring, monitoring for potential endemic agents and green-building monitoring; and
open standards-based data exchange approach that enables system interoperability for situational awareness and response.

The indoor biological detection system of the present invention comprises a hardware network of sensors, detectors, and air collectors, connected via an onsite and cloud-based software services. The system and method provide the capability to monitor, interpret, warn, and respond to possible hazard releases in single or multiple facilities. The system's hardware suite is a multi-tiered network of sensors, air sample collectors, and presumptive identification detectors, assisted by modeling applications executed online in real-time or as offline applications to detect and track a hazard release from any plausible source location while simultaneously minimizing false alarms. The system of the present invention is designed to use T&D model output (discussed below) generated “online” in real (or near-real) time for more rapid and accurate response. Online modeling and data processing increases the cost of the system but may be justified for protecting critical infrastructure/personnel when the bio threat risk is higher. A less expensive alternative is to run the models “offline” and store the results in a library for later analysis. The tradeoff is possible reduced accuracy (possible increase in missed detections or false alarms) because all threat scenarios cannot be anticipated due to the dynamic nature of the threat and the environment.

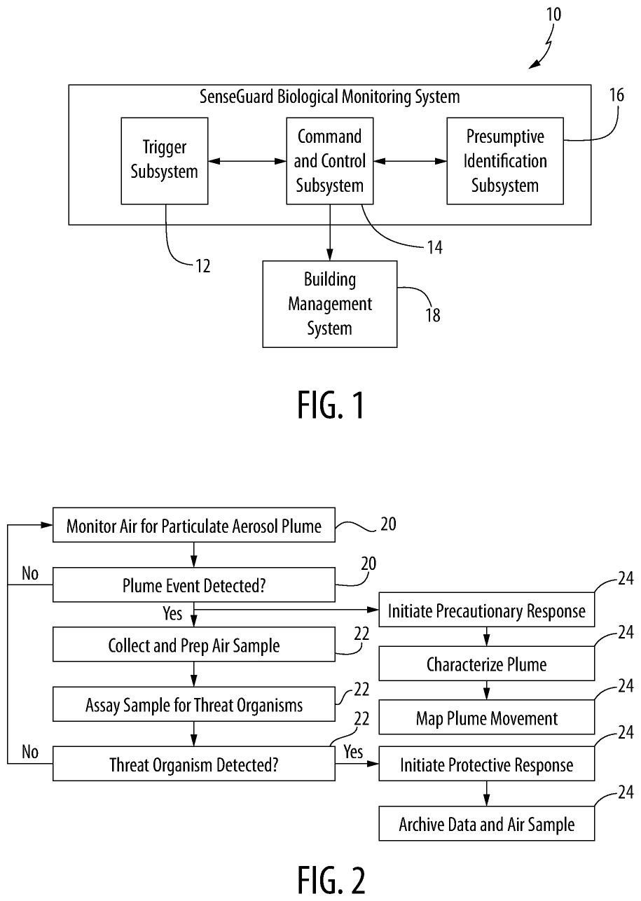

An exemplary embodiment of the indoor biological detection system 10 of the present invention is depicted in the block diagram of FIG. 1. The system 10 is comprised of three hardware and software subsystems: a Trigger Subsystem 12, a Command and Control Subsystem (C&C) 14, and a Presumptive Identification Subsystem (PID) 16. These will be discussed in detail below.

The Trigger Subsystem 12 includes particulate and/or bioaerosol sensor hardware and ancillary software that monitors the air in a building and detects aerosol plumes, i.e., particulate matter with anomalous concentrations and characteristics. Detection and characterization of a plume triggers collection of air samples, sends warning messages to local security, and initiates precautionary actions such as redirecting the HVAC system flow in a particular area of the building. Response actions that involve control of building elements such as doors, windows, and HVAC air handlers can often be implemented through issuing commands through a system interface to the facility's Building Management System (BMS) 18.

The Presumptive Identification (PID) Subsystem 16 encompasses air sampling/preparation equipment and biological assay detectors, accompanied by embedded software, that presumptively identifies bioagents present in a sample that are likely to pose a threat to building occupants. If a biological threat agent (bioagent) is identified, alarm messages are sent to building security and first responders, and high-regret (protective but operationally disruptive) actions are initiated such as evacuation of building occupants and closing doors (e.g., via a building management interface (BMS) 18).

The Command and Control (C&C) Subsystem 14 is the nerve center of the system that commands and controls components in the Trigger Subsystem 12 and PID Subsystem 16. The Command and Control Subsystem 14 is comprised of software (Application Programming Interfaces) that communicates with a large variety of sensor hardware, provides data fusion and situational awareness services, transmits notification messages to users, and issues commands to building elements through an external interface to the Building Management System 18 of a facility.

Integrated functionality of each subsystem 12, 14, 16 is depicted in the flowchart of FIG. 2. As shown, air concentration is monitored for indication of a particulate, biological aerosol plume to detect a plume event. This is accomplished by the trigger subsystem 12. If a plume is detected, the PID Subsystem 16 and the C&C Subsystem 14 are activated. The PID Subsystem collects and prepares the air sample, the sample is assayed for threat organisms to identify the threat organism. The C&C Subsystem 14 initiates a precautionary response, characterizes the plume, maps the plume movement, and, if the threat organism is identified by the PID Subsystem 16, the C&C Subsystem initiates a protective response and archives data and the air sample.

### System Function by Phase

The indoor biological detection system 10 is implemented in two distinct phases: (1) the Configuration Phase and (2) the Operational Phase. Each will be discussed in detail below.

### Configuration Phase

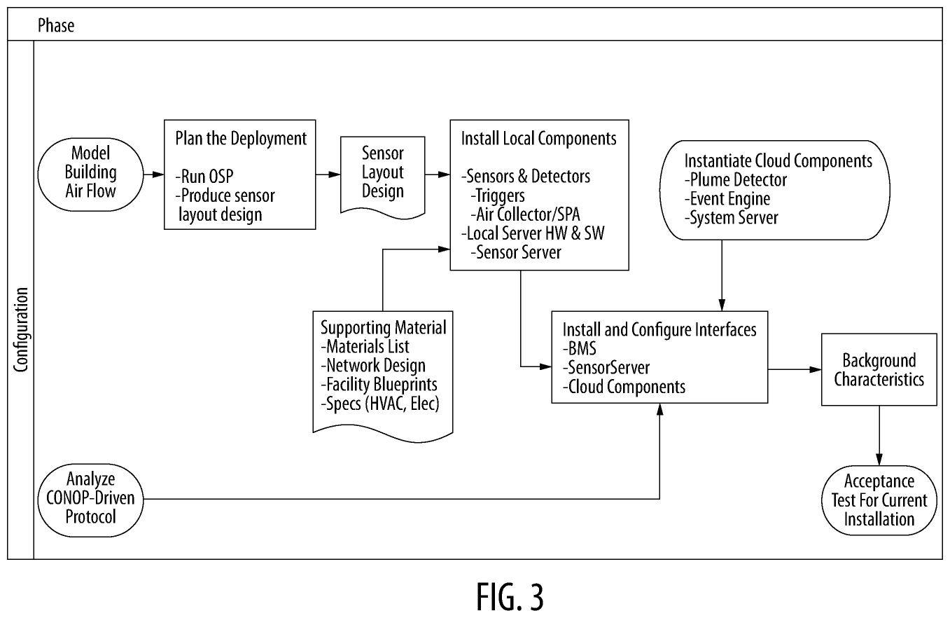

The Configuration Phase is a service performed by a system integrator to collect and enter building-specific information into the system prior to installation of the indoor biological detection system 10 in a particular building. The information determines the density and locations of sensors (including particulate sensors, air collectors, and biological detectors) to be placed in the building and how the system functions will be allocated among the specific user accounts. The system configuration process is shown in FIG. 3.

A physical building location in which the system 10 is installed is analyzed and modeled in order to define the hazard release transport and dispersion (T&D) behavior inside the building. The T&D Model is discussed below. An Optimal Sensor Placement (OSP) algorithm, also discussed below, analyzes the T&D model output to determine the number of sensors of each type and their required placement locations. The system sensors, air collectors, and presumptive identification detectors are distributed throughout the physical building location according to Optimal Sensor Placement (OSP) model recommendations. A system Bio facility cloud instance (described in detail below), containing software systems that connect the devices with the human system operators, is created, provisioned, configured to connect to the installation facility, and started up.

T&D Model:

One of the initial tasks in the Configuration Phase is construction of the T&D model used to describe and simulate air movement throughout the building, specifically of aerosolized bioagents. Modeling and simulation enable sensors to be placed where aerosol plumes are most likely to travel in a particular building. This approach is far more cost effective than using either:
Ad hoc rules such as “install a specified number of systematically spaced sensors per square foot area”
Empirical “tracer experiments” in which a series of releases are measured by a dense array of actual sensors

The modeling of indoor T&D applications can be done with CONTAM, which is an open-source computer program made available free of charge on the National Institute of Standards and Technology (NIST) website (See https://www.nist.gov/services-resources/software/contam). The CONTAM model is a multizone indoor air quality and ventilation analysis program designed to help determine airflows, contaminant concentrations, and personal exposure in buildings. Airflows include infiltration, exfiltration, and room-to-room airflow rates, and pressure differences in building systems. These airflows can be driven by mechanical means, wind pressures acting on the exterior of the building, and buoyancy effects induced by temperature differences between zones. Contaminant concentrations include the transport of airborne contaminants due to airflow, chemical, biological, and radio-chemical transformation, adsorption, and desorption to building materials, filtration, and deposition to and re-suspension from building surfaces.

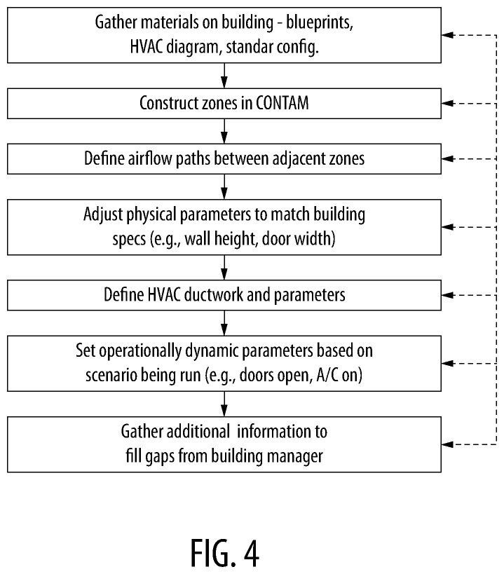

The CONTAM-derived T&D models are developed by an expert user with knowledge of commercial building design, HVAC operation, and associated physics principles such as fluid dynamics. The T&D modeling process is illustrated in the flowchart in FIG. 4. Inputs to the CONTAM model are developed by the modeler through analysis of the specific building floorplan and HVAC architecture. The building structure is decomposed into many zones (rooms, hallways, etc.) connected by HVAC elements. Certain characteristics of each CONTAM zone and HVAC element are entered by the user into the model database. The resulting CONTAM model is an executable software program that uses a set of mathematical equations to calculate the air concentration of a user-chosen contaminant (e.g., anthrax spores).

The CONTAM model executable is run many times over a series of release scenarios. Each model run simulates the T&D process within the building using a given combination of factors including release zone of origin, release amount, and release duration. The series of CONTAM runs, called an ensemble, generates a large batch of data files, each containing predicted bioagent concentrations by zone and time step. The generated Model Release Files, which provide the predicted spatial-temporal pattern of bioagent hazard flow from a release in any area (zone) of the building, are subsequently stored in a library or database. The modeled concentration data are used in two special algorithms: 1) Optimal placement of sensors and 2) Detection of biological hazard plumes. Details of both algorithms are described in the following sections.

Optimal Sensor Placement

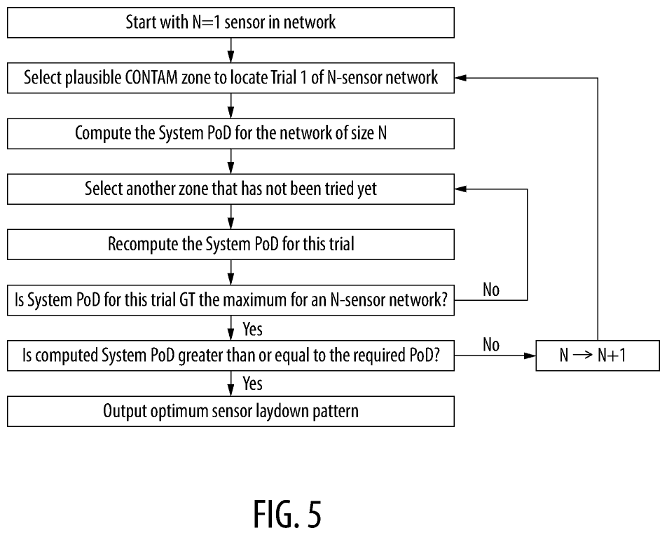

The optimal sensor placement configuration for a given building, referred to as the “laydown,” is determined by a software program, i.e., an Optimal Sensor Placement (OSP) module. The OSP module finds the least expensive arrangement of sensors that meet the specific requirements, usually the system-level Probability of Detection (PoD) specified by the customer/buyer. The OSP module uses a “forward selection” optimization algorithm which is depicted in FIG. 5.

This OSP algorithm begins with one randomly selected zone in which a sensor can plausibly be placed. The System PoD is measured, and then sensors are systematically added and System PoD remeasured. Whichever add-one-sensor laydown gives the highest System PoD is chosen as a new “base” laydown. The process iterates, using this new base laydown as a set to experimentally add one sensor from and re-evaluate the System PoD. This process continues until a laydown is reached that is greater than or equal to the required System PoD, but from which no sensor can be removed without causing the resulting laydown to go below the required System PoD.

Non-limiting examples of the types of biological sensors that are compatible with the various tiers of the present system are described in Table 1, below.

TABLE 1
Examples of Off-the-Shelf Devices for the Indoor Biological Sensor System

| Device | | | |
|---|---|---|---|
| Type | Example | Tier | Description |
| Particulate | HabitatMap | 1 | Low-cost, palm-sized air quality monitor that |
| Sensor | Airbeam2 | | measures hyperlocal concentrations of particulate |
| | | | matter in the air |
| Particulate | TSI AeroTrak | 1 | Measurement of particle counts by at various size |
| Sensor | RPC | | ranges using laser technology; used in a variety of |
| | | | industrial settings |
| Bioaerosol | ATI Polaron | 2 | Real-time detection of airborne biological agents |
| Sensor | F10+ | | (spores, toxins, viruses, and bacteria) with high |
| | | | sensitivity and low false alarm rates |
| Bioaerosol | FLIR IBAC 2 | 2 | Bio-detection sensor that can support continual 24/7 |
| Sensor | | | air monitoring or can be set to targeting sampling windows |
| Bioaerosol | Zeteo Tech | 2-3 | Detection system includes a UV Laser Induced |
| Sensor | BioFlyte z200 | | Fluorescence trigger sensor and a time-of-flight |
| | | | mass spectrometer for threat identification |
| Air | InnovaPrep | 3 | Light weight, portable, dry filter air sampler with a |
| Collector | ACD-200 | | unique rapid filter elution kit; ideally suited for |
| | Bobcat | | collection of bioaerosols and particulate matter |
| Biological | Biomeme | 3 | Portable, multiplex real-time Polymerase Chain |
| Identifier | Franklin | | Reaction (PCR) Thermocycler that amplifies and |
| | three9 | | identifies biological species of interest from DNA or |
| | | | RNA from prepared samples |
| Biological | Smiths | 3 | Portable bioaerosol collection and identification |
| Collector & | Detection | | system; allows up to 16 agent-specific biosensors |
| Identifier | BioFlash | | using antibody and bioluminescent molecules |

Operational Phase

In the operational phase, the indoor biological sensor system 10 of the present invention monitors the air inside the building/facility and provides warnings and alarms to safety personnel and ultimately the building occupants and other stakeholders.

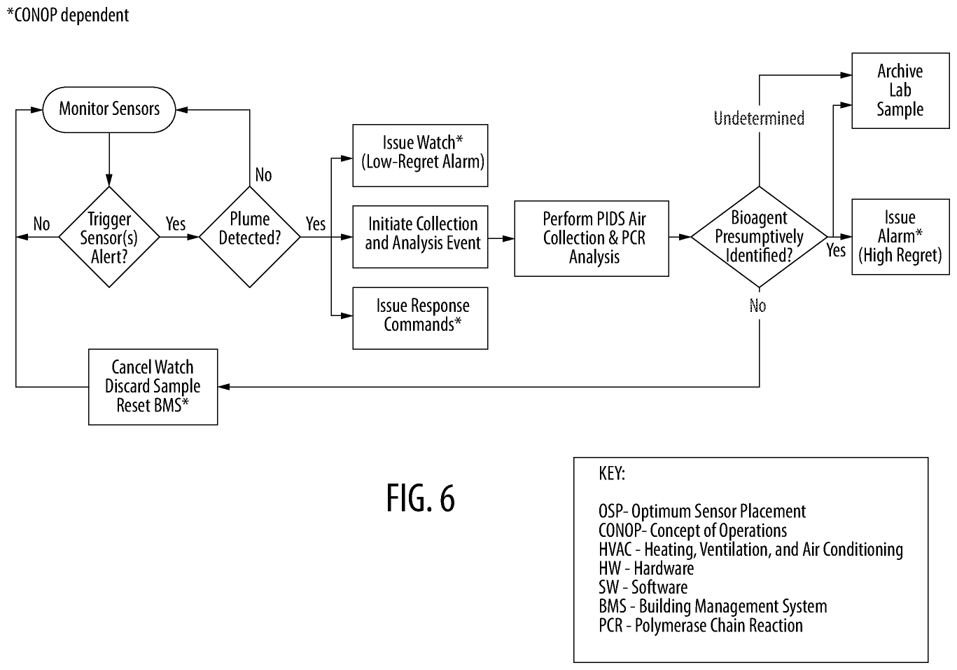

The logic flow inherent in the Operational Phase of the indoor biological sensor system and method 10 is depicted in FIG. 6.

There are several activities that may be going on while indoor biological sensor system 10 is in operation at an installation/facility. These include:
Plume Detected—Plume Detector defines events in which the indoor biological sensor system has detected a plume within the installation. The system does this by constantly running an algorithm, which monitors the outputs of 1^{st} tier air particle sensors and determines if the particulate counts, sizes, distribution, and movement match a predicted (by the CONTAM indoor T&D model) plume pattern stored in the Model Release File library (described above). If so, the system automatically generates a Plume Detection alarm. Once a plume has been detected, the system automatically escalates to a 2^{nd} tier of sensors and further characterizes the plume. Plume Detection exercises Tier 3 of the system.
Bioagent Presumptively Identified—Biohazard Presumptively Identified is an event in which the 3^{rd} tier Field Screening Device of the indoor biological detection system 10 has positively identified a specific bioagent because of an air sample analysis having been triggered by the Plume Detected event. During the exercise of the 2^{nd} tier bioaerosol sensors, the system automatically determines the most likely location of the source of the potentially hazardous biological release and causes the air collector to collect a sample. The most likely source location is determined using a statistical algorithm that matches trigger sensor alarm patterns with all simulated hazard releases. The source of the simulated release that most closely matches the trigger alarm pattern is deemed the most likely source location. The sample output from the air collector is automatically prepared for analysis and passed to the 3^{rd} tier Field Screening Device. The Field Screening Device considered as the gold standard in the industry is the real-time Polymerase Chain Reaction (PCR) detector. The 3^{rd} tier Field Screening Device decides either the presence or absence of the biological hazard and provides notifications. This notification, or alarm, allows for mitigation and response activities to be either automatically executed, such as HVAC shutdowns, or executed by safety personnel, such as an evacuation. Several internal and external responses need to be initiated, with the system 10 providing initial coordination of these.
Post-Release Forensics—The sample preparation function of the indoor biological detection system 10 automatically archives a sample in a removable vial after any air collection event. This function is provided so that in the event of an actual hazardous release, the archived sample will be available to authorities for additional testing and verification. The system also stores the system logs so that all post-event reviews and analysis of the event can be done by authorities.

### Hazard Response Strategy

The indoor biological detection system and method's 10 hazard response strategy uses a three-tiered approach to detect a release and determine if the release is hazardous. The Trigger Subsystem may contain one or two tiers of sensors of different types. Tier 1 could be a network of particulate sensors, monitored by a software module called Plume Detector (PD) Module used to identify plumes. The Plume Detector Module is discussed below. If additional confidence in aerosol threat detection is required, an array of bioaerosol sensors that detect biological organisms in the particulate plume could be configured as Tier 2. An analytics software application is integrated with Tier 2 sensors, comprised of an Airflow model (CONTAM), discussed above, and a analytics program called the Source Location module. The probable source location and airflow model, along with the Tier 2 bioaerosol sensor data, are used to classify the particulate plume as biological or non-biological. Tier 3 is the Presumptive Identification Subsystem and consists of an Air Collector, a Sample Preparation Accessory and a Field Screening Device used to sample a suspected hazardous release and presumptively confirm or deny that the sample contains a known hazard.

Each Trigger Subsystem 12 sensor is a portable device that counts the number of very small (a few micrometers) particles in the air. The sensor pulls aerosol into a chamber and then the device counts the particles that are in pre-set size categories called size bins. The limits of each size bin are controlled in firmware onboard the trigger sensor device. Bioagent particles typically have a very well-defined size distribution, so the indoor biological detection system and method tunes the size bin configuration to have the greatest chance of detecting specific bioagents. The output (counts per unit volume of air) of the Trigger Subsystem sensors are sent wirelessly to the indoor biological system's sensor server located within the facility installation and then relayed to another server in the cloud to be analyzed by the Plume Detector and Source Location modules. These modules are further described in the following subsections.

Plume Detector Module

The Plume Detector algorithm is, at its heart, a volumetric pattern-matcher across time and space. Its function is to monitor ongoing sensor alerts and see if, taken in total across a window of time, they add up to a possibly hazardous plume. It does this by comprehensively tracking “Release Theories” and sounding the alert if any release theory is matched too well by actual data to be considered pure accident.

A “Release Theory” in this context is the thesis that a release happened at a particular timestep, originating from a specific zone. Each release theory can thus be keyed on its starting time and originating zone (release zone). A release theory is a time-series of expected sensor alerts over time fora given release zone, calculated by looking at the Model Release File for that release zone and, at each timestep, listing out trigger sensors in the current laydown that correspond to zones at that timestep which are modeled to be above threshold concentrations. It is thus a trace of a plume as it moves through a facility and is sensed by our network of trigger sensors. It is implemented as a linear Hidden Markov Model where the states correspond to relative timesteps from the beginning and each state contains the expected set of alerting sensors at that timestep. If the CONTAM model is correct, each possible plume should have a unique trace across time and be distinguishable as a), a plume and not a benign “false alarm”, and b) different from all other legitimate plumes.

As a Release Theory is tracked during Plume Detector operations, a time counter is incremented (per timestep) and several scores are kept. It is possible to have many existing release theories which are all based on the same model release file, but which differ in their starting times (and time counters). For example, one release theory might track a possible release starting from CONTAM zone #1 three timesteps ago, another Release Theory might track a possible release from zone #1 two timesteps ago, and a third release theory might track a possible release from zone #1 just one timestep ago. They each are trying to match against the same time-series of sensor alerts, but each is at a different timestep point along that time-series. This approach allows plume detector to handle a release happening at any point in time.

A Sensor Snapshot is a composite record of the observed alert statuses of all trigger sensors during the last timestep. The Plume Detector collects this information at each timestep from the Model Release File library. The sensor alert records in the library are at a finer temporal granularity than the current Plume Detector timestep; if a particular trigger sensor alerted at any time within the span of a Plume Detector timestep, it is considered to have alerted for that timestep.

Release Theories are scored according to how well they match observed data. At each timestep, the current record of sensor alerts (the Sensor Snapshot) is sent to a release theory for scoring. The following scores are kept:
CUSUM—This is a measure based on the number of sensors are alerting above the expected number of “falsely alarming” sensors. This measure is tracked across time and does not count specific expected vs observed zones, just a more general number above particulate “background” during a particular timestep.
HMM/Binomial Probability—This is a more sophisticated measure that counts the differences between the observed alerting sensors in the latest sensor snapshot and those that were expected to alert on this timestep if an actual release was going on. The “Hamming distance” (number of sensor alert differences between expected and observed) is counted and fed into a binomial probability calculation which is the scoring method for our HMMs. This score is always on a [0, 1] scale.

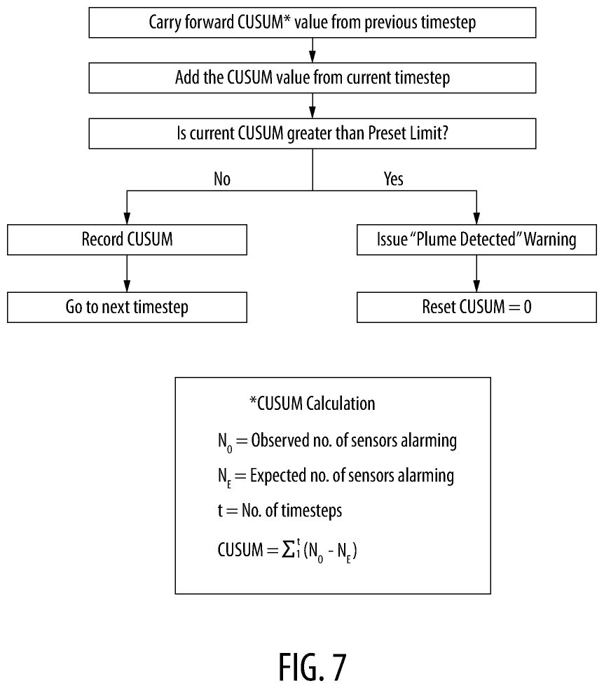

The Tier 1 function of the Plume Detector is to recommend if a “Plume Detected” warning should be issued to the facility. The relatively simple logic flow of the Plume Detected event calculation is presented in FIG. 7. Such a warning might result in taking certain actions to contain a possible hazardous release, such as changing HVAC settings or closing doors. The Plume Detected Warning is issued when the number of alarming sensors results in a CUSUM score that exceeds a statistically determined limit. This limit is dependent on the reliability of the chosen trigger sensor and two competing risks: a) health impact on the facility population if an actual bioagent were released and b) effort and resources expended to characterize and disconfirm the suspected plume if the sensor alerts are not caused by a bioagent release.

A “Plume Characterized” event is the Tier 2 function of the Plume Detector. This is a conclusion about the nature of a suspected plume, such as where it started, when it started, does it contain biological organisms, and what the best location to collect a sample would be. When the Plume Characterized determination is made by Plume Detector, this signals to the rest of the indoor biological detection system to begin the Air Sample Collection/Presumptive Identification process.

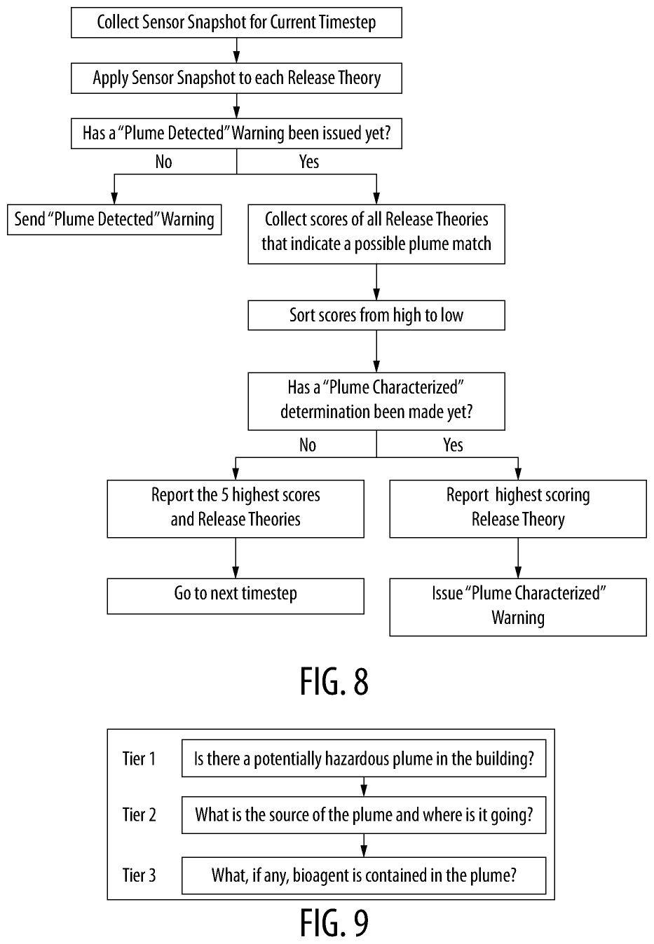

The logic flow of the Plume Detector Tier 2 event determination during each timestep is depicted in the flowchart FIG. 8.

### Relationship Between Tiers of System Components

In the biological monitoring system architecture, three tiers of components generate data streams that are fused and analyzed with the aid of Artificial Intelligence and Machine Learning (i.e., intelligent software) methods. The three tiers answer specific questions necessary to respond to a biological event. (See FIG. 9) The three tiers represent information sources, e.g., different sensor types, from which data are analyzed in a specific order. The occurrence of a particular physical phenomenon will cause anomalies in the data, such as sensor output exceeding a threshold value, a specific pattern of biological detections that indicate a threat, or other statistically significant result. An anomaly occurring at a given information tier is designated as an event, which provides an independent piece of evidence that the system uses to detect, locate, characterize, and understand possible biohazard threats.

The order of the tiers (as well as events) is chosen to enhance the timeliness and cost effectiveness of the system to detect and identify a hazardous biological aerosol release in the building. Lower order tiers/events provide information relatively quickly and inexpensively but are less accurate. For example, air quality monitors (particulate counters) are typically chosen as Tier 1 sensors. An example of a Tier 1 sensor is the AirBeam2, a commercial off-the-shelf air quality monitor manufactured by HabitatMap (https://www.habitatmap.org/airbeam/buy-it-now). These sensors have a very low purchase price (less than $250) and operating cost (require occasional cleaning) and can rapidly detect (in a few seconds) an anomalous increase in particles of a specific size range (e.g., 1-5 micrometers).

Higher level tiers/events provide more specific information to determine if the aerosol plume contains biological material but are more expensive to purchase and operate. An example of a Tier 2 sensor is a Polaron F10+ sensor which can be purchased off-the-shelf from Air Techniques International (https://www.atitest.com/products/polaron-f10-real-time-bioaerosol-sensor/) for several thousand dollars. Finally, a field screening device is required to presumptively identify the biological agent. For example, a Real-Time-Polymerase Chain Reaction (RT-PCR) detector such as the Franklin three9 (https://shop.biomeme.com/products/franklin-real-time-per-thermocycler) can be purchased off-the-shelf for Tier 3. Obtaining this higher level of information requires a much higher purchase price (about $10,000) than the Tier 1 particulate sensor, an air collector and sample preparation apparatus (several thousand dollars), a supply of consumable chemical reagents that must be replenished for each run (around $150), and a longer run time (approximately 30-45 minutes).

Using Tier 1 information, a single individual particulate sensor can rapidly detect an anomaly in its immediate locale, but it does not necessarily represent a hazard unless there is a significant temporal pattern of sensor detections (called a plume event). Detection of a plume event is accomplished by a statistical method that detects a significant increase in the number of sensors that show concentrations of particles (of a selected size range) significantly above a background level of particulate concentrations in each area of the building. Pattern detection requires a higher density of sensors distributed across areas of a building. The distribution is optimized to use as few sensors as necessary to define the area of the building that the plume has affected. When a Tier 1 plume detection event is detected a Biohazard Watch (Alert) is issued to the system user, warning them of potential danger to health in the building. In addition, the event automatically initiates the collection and analysis of Tier 2 information to characterize and localize the threat.

The purpose of Tier 2 sensors in the system architecture is to determine the biological threat content of the plume area, i.e., the probable source of the hazard release and the area of the building that is likely to be affected by the threat. This is accomplished by analyzing information obtained from Tier 2 sensors as well as simulations using repeated runs of an indoor T&D model such as CONTAM. A machine learning method is used to locate the most likely source of the release by searching the database of model runs that most closely matches the observed trigger sensor events at each point in time since the release. The particulate concentrations from the best matching model run are then plotted on a map to inform the system user of the areas in the building that are most likely to be affected by the hazard. The best model run information is also used to predict the amount of a target DNA sequence needed for the PCR thermocycler to identify a specific bioagent, if the sample truly contains bioagent. If the particulate concentration at one or more air sampler locations is sufficiently large, and if Tier 2 bioaerosol sensors in or near the plume indicate biological organisms are present, Tier 3 collection and analysis will be automatically triggered by the system.

The purpose of Tier 3 of the biological monitoring system is to identify whether a bioagent (hazardous biological organism) is present in an aerosol sample of air taken from the hazard plume. The sample must first be collected, liquified, processed, and prepared for bioassay. The biological identifier performs a series of simultaneous (DNA) assays that determine whether the contents of air sample match one or more organisms from a predetermined set of biohazard agents. When a biohazard is (presumptively) identified, a Bioagent Identified (Alarm) is automatically issued to the occupants of the building, as well as a notification sent to building safety and potentially, first responders.

Two tiers of sensor information are considered the minimum viable configuration to provide reasonable protection of a building occupants from the release of biological threat agents. Three or more sensor tiers will provide additional confidence in detection decisions that may be required for protection of critical assets. The system architecture allows additional tiers of information to be added as necessary to provide better awareness and understanding of the threat. Addition of new tiers may be desirable to further lower the risk to the occupants as a function of the total cost of system operation. For example, video feed from a closed-circuit camera could be analyzed as further confirmation or disconfirmation of the human activity associated with a biological agent release. New tiers of sensors and algorithms, and the rules that control their execution can easily be incorporated in the event detection capability (called the Event Engine) of the indoor biological detection system. The Event Engine is the high-level decision support mechanism that coordinates the input and output of the information tiers and communicates the decisions in the form of standard messages, e.g., warnings, alerts, and Building Management System Commands.

Event Engine Capability

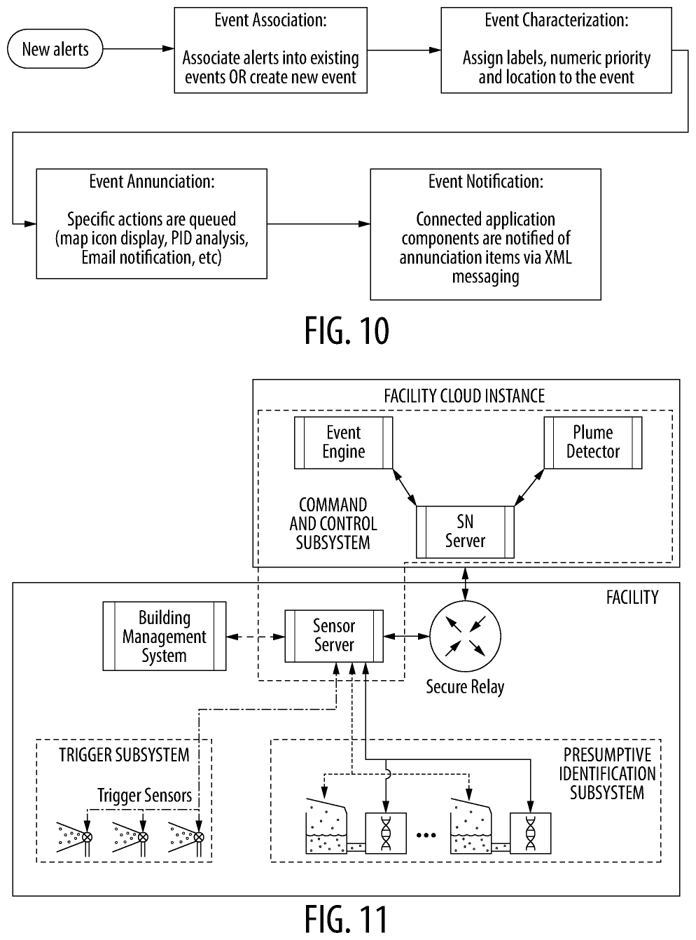

The Event Engine (EE) capability is the central software component in the indoor biological detection System Server. The EE is a rule-based process that associates sensor alerts and Plume Detector (PD) warnings into events. It characterizes events as threats and requests automated actions (bio confirmation, e-mail sends, audible alarms, display of events) to connected application components. The high level data flow of the EE is illustrated in FIG. 10.

There are two main functional data flows occurring within the EE. One is association of alerts into events. Second is the event characterization/annunciation. The following describes these two processes.

Alert Association

For the indoor biological detection system, there are currently two types of sensor alerts. One is from the Plume Detector (PD) component (i.e., the Trigger network) and the other is from the Presumptive Identification Detector (PID). Trigger sensor alert data is inspected by the Plume Detector component. This means that as trigger sensors enter an alert state, ‘event’ objects are not being created in the system.

Alerts from PD are stored in a specific set of PD database tables. PD tags an alert with a special attribute which informs EE that the alert is part of the analysis of the same plume release scenario. EE associates PLUME alerts based upon a specific tag. This association rule is defined by the database configuration of the detection class named ‘PLUME’.

Alerts from the PIDs are stored in a specific set of PIDs database tables. If the PID alert was a result of an automated analysis, the indoor biological detection system Server will tag the alert with the system event ID which triggered the sample. EE inspects this value to decide whether to associate that alert into the existing trigger event. If the PID alert is a result of a manual sample analysis run, the indoor biological detection System Server will assign a value in the alert which will cause EE to create a new event. This association is defined by the PID detection class.

As the association process updates or creates new events, it signals the event characterization process that there is new work to do using a named ‘OS event’ as the inter-process signaling mechanism.

Event Characterization

The event characterization process is driven by two main sources. One is an Extensible Markup Language (XML) file linked to the Site of the event which contains the rule set for assigning the event characterization string (e.g., ‘Positive bio release’) and the event priority (1 to 10 numeric level where 1=high threat and 10=non-threat). The other is an annunciation rule set in the database which configures which automated actions to take based upon the event properties.

The current event characterization config file for the indoor biological detection system 10 is summarized in Table 2 below.

TABLE 2
Event Characterization Conditions and Priorities

| Characterization | Priority | Condition |
|---|---|---|
| Bio plume preliminary | 9 | PD has reported some alerts but has not yet provided an |
| | | alert with a plume origin (release in a CONTAM zone). |
| Bio plume located | 5 | PD has reported an alert with a CONTAM zone |
| | | identifying origin of the plume. |
| Positive bio release | 1 | A PID sensor has reported a positive threat detection on |
| | | sample analysis. |
| Negative result on bio | | A PID sensor has reported a negative result on sample |
| presumptive identification | | analysis. |
| Error result on bio | 5 | A PID sensor encountered an error while performing |
| presumptive identification | | sample analysis. |

The conditions above are defined by database query statements. They can be updated at any time to change how future events are characterized. Any data present inside the indoor biological detection system Server database can be used in the rule set driving the event characterization (e.g., say in the future we want to condition positive declaration based on time of day, environmental sensors, etc.).

The event priority values in the table above were chosen arbitrarily to tag a threat level to the event. The values affect the color-coded label on the indoor biological detection system client User Interface (UI) only; they do not drive any other data flows in the system.

The event characterization strings in the table above are used by the annunciation rules to determine which automated actions to take.

Event Annunciation

Any time an event is created or updated, the annunciation rule set is examined to see what actions need to be requested. The system is flexible and can be configured with any number of rules to support any specific Concept of Operation (CONOP). The indoor biological detection system is configured with two simple rules:
1. On a ‘Bio plume located’ event, request a ‘Identify Agent Presence—BIO’ action. The indoor biological detection system 10 Server subscribes to handle this type of action and will be sent an XML message to initiate the sample analysis ‘job’.
2. On a ‘Positive bio release’ event, request an ‘Email’ action. The Emailer service connected into the indoor biological detection system 10 server is subscribed to handle this type of action and is notified with the request to send an email. The recipient(s), subject, and body of the email are configurable (EvAnnun.exe on the server is used to configure all annunciations). Appended to the body of the email is a web page address linking back to a web application. The recipient can click the link to launch the Web UI of the app. This web address is configurable in the indoor biological system database.

Other rules may be configured later (sound audible alarm, display event on UI, etc.) but the two actions above are the main ones for system operation.

Optimal Sensor Placement Capability

The Optimal Sensor Placement (OSP) method is designed to be run during the Configuration Phase of a system installation into a facility. This module attempts to discover an optimal (in the minimal cardinality sense) laydown of sensors that meet System Probability of Detection (PoD) requirements while covering all perceived plausible release conditions. “PoD” should be understood as “Probability of (Plume) Detection” and reflects the percent of time that a plume with some identifying characteristics (e.g., source location, source term, etc.) is correctly weighed as the highest likely explanation (distinct from other competing plume possibilities or the possibility of “no release at all”) for a given time-series of observations.

This iterative optimization method begins with a sensor in every plausible Sensor Location Zone, measures the System PoD, and then systematically removes sensors and remeasures System PoD. Whichever remove-one-sensor configuration scores the best System PoD while remaining above the required System PoD is chosen as a new “base” laydown, and the effort repeats, using this new base laydown as a set to experimentally remove one sensor from and re-evaluate the System PoD. This iteration continues until a configuration is reached that is above the required System PoD, but from which no sensor can be removed without causing the resulting configuration to go below the required System PoD.

The OSP method depends on the following input information already being determined/executed:
1) Facility modeling that identifies and demarcates all plausible Sensor Location Zones and
2) T & D modeling (CONTAM is one such T&D model that can be utilized) that captures the concentration profile (in terms of time and sensor locations) for each plausible plume
3) T & D modeling results being formatted into text files, one file per permutation of {Source Terms X Operational Conditions X Source Location Zones}
4) Minimum and maximum number of sensors allowable
5) Minimum sensor threshold given the alarm requirements

The OSP generates a final results file that lists the minimal set of sensors that meet the System PoD within an established timeframe. If no such solution exists, the closest solution (according to some measure and with respect to certain constraints having priorities over others) will be identified instead, with appropriate messaging indicating the shortfall(s). Alternatively, if no constraint-fulfilling solution is discovered then an output saying as much will be generated.

### Indoor Biological Detection System Software Design

Facility Cloud Instance

Most of the software for an indoor biological detection system instance operates within a cloud architecture, which is flexible according to demand and more robust under stress. There is one Facility Cloud Instance for each supported installation (facility or building). Each instance is a single copy of software running on a single (physical or virtual) computer server that contains modules to receive and analyze sensor alerts from the installation; these modules will be detailed later. A facility cloud instance is connected via a Secure Relay to the Sensor Server, physical devices (sensors, detectors, air collectors), and the Building Management System, all located in the facility (See FIG. 11).

Facility Cloud Components

The components deployed in the Facility Cloud Instance include software components, a relational database and web services. References will be made to the sensors, collectors, detectors, and the Sensor Server, but those elements will be more properly detailed in the Facility-Specific Components section below.

TABLE 3
Indoor Biological Detection System Facility Cloud Components

| Component | Purpose |
|---|---|
| Indoor Biological Detection | Collects and collates trigger sensor alert statuses from the |
| System Server | Sensor Server; Collects messages for devices from the Sensor |
| | Server; Initiates device maintenance, adding, dropping; |
| | Records all command and control actions; Serves UI |
| | webpages for all Facility level users for the installation; |
| | Provides all Web Services for communication between all |
| | software components. |
| Event Engine | Rules engine for contextualizing sensor alerts within the |
| (EE) | operational reality of the facility; Creates Event records which |
| | constitute a trace of indoor biological detection system's |
| | response to a possible hazard release. |
| Plume Detector | Detects plumes by collecting trigger sensor alerts at regular |
| (PD) | intervals and comparing them against T&D-modeled sensor |
| | alert patterns; Raises alarm if plume is detected and provides |
| | updates about most likely source location and which air |
| | collector/presumptive identification detector is best positioned |
| | to sample and confirm a hazard release. |
| Indoor Biological Detection | Creates/Reads/Updates/Deletes the following: Facility |
| System Database Server | Supervisor actions; Sensor/detector laydown information; |
| | Recent archive of all trigger sensor readings; All Events |
| | formed by the Event Engine in response to its rules; PD |
| | messages for a possible hazard release. |

Facility-Specific Components

This section describes the components deployed in the Installation or Facility, which include the Sensor Server software component, a secure relay (router), and all trigger sensors, air collectors and presumptive identification detectors in the facility.

TABLE 4
Facility-Specific Components

| Component | Purpose |
|---|---|
| Sensor Server | Handles all communications to/from all indoor biological detection system |
| | devices (trigger sensors, air collectors, presumptive identification detectors); |
| | Passes messages to/from the Building Management System (if available); |
| | Communicates all device statuses to the indoor biological detection system |
| | Server in the Facility Cloud Instance via the Secure Relay; Logs all raw device |
| | state data to its local file system for archive and forensic analysis. |
| Secure Relay | Configured as a VPN appliance to connect the facility being protected to the AWS |
| | cloud environment hosting the indoor biological detection system application |
| | software; Provides secure connection between Sensor Server running in the |
| | facility and the indoor biological detection system Server running in the cloud. |
| Trigger Sensor | Fast and inexpensive, commercial-off-the-shelf (COTS) air quality sensors that |
| | detect and count particles fitting a certain size range or particle size distribution; |
| | Possess alert capability based on particle counts exceeding a given threshold. Air |
| | quality (i.e., particulate) sensors may be supplemented by bioaerosol sensors |
| | that use laser-induced fluorescence or comparable technology to detect the |
| | presence of biological material in the aerosol. |
| Air Collector | Collects an air sample for a specified amount of time and elutes the sample into |
| | fluid that can be delivered to the Presumptive Identification Detector for PCR analysis. |
| Sample Preparation | Autonomous sample preparation and delivery to a PCR detector; Stores archive |
| Accessory | sample for laboratory or forensic analysis. |
| Presumptive Identification | Receives the eluted sample from the Air Collector and performs a PCR analysis on |
| Detector | it to presumptively identify if the sample contains a hazardous agent or not. |

### Building Management Systems

The indoor biological detection system can be integrated to interface with a Building Management System (BMS) via the secure connection to the instance of Sensor Server running in the facility being monitored. Command and control messaging will be sent across this communication link. Depending on the type of BMS present in the facility, Sensor Server can either perform the command-and-control actions directly to HVAC devices or interface with proxy software to communicate with them. The design purpose of the Sensor Server command and control is to implement Biothreat mitigation actions as specified by the building owner.

### Indoor Biological Detection System Hardware Design

The indoor biological detection system is comprised of a combination of both hardware and software components. This section will focus on the indoor biological detection system hardware design and its integration into the Trigger Subsystem (TS) 12 and the Presumptive Identification (PID) Subsystem 16.

The indoor biological detection system TS is comprised of two software components (OSP Tool, the PD including the Source Location Algorithm) and one or two types of hardware components (Trigger Sensors that measure particulate matter or biological material concentrations). The functional requirements of the TS are to consistently monitor an indoor space for the presence of a biological threat release and to distinguish threat releases from background. The basic design requirements of the TS are to provide fast response time, excellent sensitivity, a low false alarm rate, modularity, and low cost. The individual trigger sensors have a low initial hardware unit cost. The TS combined performance is greatly improved as compared to a single sensor by utilizing the OSP and PD modeling/algorithmic components.

The indoor biological detection system PIDS is comprised of three main hardware components, an Air Collector (AC), a Sample Preparation and Delivery Accessory (SPA), and a Field Screening Device, typically a PCR presumptive identification detector (PID). The purpose of the PID is to autonomously identify a bioagent as one of a set of biological threat agents that are considered the most dangerous. The PID functions to autonomously collect, prepare, and analyze an air sample for biological threat agents within 60-90 minutes. The subsystem design requirement is to provide an extremely low false positive rate and high probability of detection.

For the integration of the indoor biological detection system hardware components, a modular, open systems approach to the design is applied. This approach allowed the system to leverage commercial-off-the-shelf (COTS) products for certain components in the system. This aids in reducing system hardware costs and providing the flexibility to facilitate future technology refreshes. The main COTS components of the indoor biological detection system are air quality monitor sensors which comprise the system's TS, and a PCR presumptive identification device used to presumptively identify or deny the presence an actual biological threat. Both of these COTS components had minor modifications in order to function as components of the indoor biological detection system. The COTS PCR presumptive identification device required minor modifications to allow it to operate in a fully autonomous mode and be commanded by the indoor biological detection system controlling software. The TS requires form and fit modifications to allow for facility installation and operation (e.g., wall mounted installation, communications modalities).

Trigger Sensors

Trigger sensors are an integral part of the trigger subsystem design. The trigger sensor serves as the first-tier sensing system that is designed to be inexpensive modular COTS, yet when coupled with of the rest of the trigger subsystem provides high fidelity detection to aid in the minimization of false alarms. The trigger sensors have been selected based on the ability to distinguish between certain particle size channels as well as detecting fluorescence characteristics indicating the presence of organic (biological) material. Monitoring particles within the specified size range and fluorescence allows the system to distinguish a Bio-threat release from non-threat particulate. Data from the trigger sensors feeds the PD to distinguish a threat release from background.

Air Collector (AC)

The purpose of the air collector (AC) is to autonomously collect and elute air samples for analysis by the presumptive identification detector. The AC is designed to be capable of collecting at least eight sequential collections without replacing consumables, and without degradation of collected biological samples. In addition, the AC was designed to be a modular design component that can also support manual analysis by any PCR or lateral-flow immunoassay (LFI) device.

Sample Preparation Accessory (SPA)

The purpose of the SPA is to provide fully autonomous collection of eluted samples, preparation (spore lysing), and delivery of biological samples. In addition, the SPA is responsible for the storage of both archive sample for reach back analysis and waste sample material for proper disposal.

Presumptive Identification Detectors (PID)

The purpose of the presumptive identification detector (PID) is to provide an indication as to whether the first-tier alert is a biological threat. For the current indoor biological detection system application, the PID selected is a PCR device.

Communication Network

While the communications network is not part of the indoor biological detection system design, it is a critical component of the overall architecture. The indoor biological detection system has been designed to easily integrate with most standard facility level networks via a secure Local Area Network (LAN).

Secure Relays

A secure relay is used to protect the indoor biological detection system facility installation from unauthorized intrusion.

User Interface

A User Interface (UI) allows a user of the system to view the state of the facility and perform specific actions. The UI provides a map-based, schematic view of each level of the facility, depicted side-by-side over the actual facility on the map.

The indoor biological detection system is a system created upon a flexible, scalable, and modular system architecture that adapts as the component state-of-the-art evolves. The indoor biological detection system is capable of adapting to new and emerging biological threats fora variety of indoor venues, such as office buildings and conference centers. It is also capable of incorporating information from sensors for traditional and non-traditional chemical agents, toxic industrial chemicals, explosives, and radiological threats. We have created an autonomous system with application across a broad spectrum of threats for complete threat alert, identification, and response.

The indoor biological detection system has advantages including:
1) Capable of integrating N tiers of sensor information and analytic processes, each tier building the confidence of bioagent detection on the previous tier
2) Fusion of large volumes data from multiple types of sensing devices including laser particulate matter sensors, fluorescence detectors, PCR thermocyclers, and mass spectrometers
3) Incorporation of very low-cost trigger sensors that reduces total cost of ownership (acquisition, operation, and maintenance)
4) Innovative algorithms for optimized sensor placement, plume detection, and plume characterization, which maximizes system probability of detection and minimizes false positive rate
5) Fully automatic process from monitoring to sample collection to bioagent identification, issuing warnings and alarms
6) Processing time under one hour from plume detection to bioagent identification, which is a major improvement over current systems' time-to-detect and identify, which is 24-48 hours.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. A method for indoor biological detection of a monitored space, comprising:
(a) collecting and entering monitored space information to determine density and location of sensors for monitoring air in the space for an aerosol plume,
(b) distributing the sensors throughout the monitored space;
(c) monitoring the air and detecting and characterizing a plume event;
(d) determining a source location;
(e) collecting and preparing an air sample upon the detection of the plume event;
(f) assaying the air sample to identify a hazardous release utilizing at least one field screening device;
(g) initiating a precautionary response for the hazardous release;
(h) characterizing the plume as biological or non-biological; and
(i) initiating a protective response.

2. The method for indoor biological detection of a monitored space of claim 1, wherein the step of determining a source location includes analyzing and modeling data collected to define hazard transport and dispersion behavior to determine a contamination map.

3. The method for indoor biological detection of a monitored space of claim 1, including a step of mapping and monitoring dynamic plume movement within the monitored space.

4. The method for indoor biological detection of a monitored space of claim 1, wherein the step of distributing sensors includes distributing a first tier of particulate sensors and a second tier of bioaerosol sensors.

5. The method for indoor biological detection of a monitored space of claim 1, wherein the steps of collecting and preparing an air sample and assaying the air sample are accomplished with a presumptive identification subsystem.

6. The method for indoor biological detection of a monitored space of claim 3, wherein the steps of initiating a precautionary response for the hazardous release, characterizing the plume, mapping plume movement within the monitored space, and initiating a protective response are accomplished with a command and control subsystem.

7. The method for indoor biological detection of a monitored space of claim 1, wherein the sensors include sensors selected from the group of particulate sensors, bioaerosol sensors, air collectors, and field screening devices.

8. The method for indoor biological detection of a monitored space of claim 1, including analyzing the transport and dispersion behavior utilizing a sensor placement algorithm to determine a number and type of the sensors and placement within the monitored space.

9. The method for indoor biological detection of a monitored space of claim 1, wherein the step of analyzing and modeling the monitored space to define the hazard transport and dispersion behavior utilizes an open source CONTAM computer program.

10. The method for indoor biological detection of a monitored space of claim 1, wherein including a step of presumptively identifying material from a hazardous release utilizing a field screening device.

11. The method for indoor biological detection of a monitored space of claim 10, wherein the field screening device is a Polymerase Chain Reaction (PCR) detector.

12. The method for indoor biological detection of a monitored space of claim 1, wherein the step of initiating a protective response includes a response selected from the group consisting of changing HVAC settings, closing and opening doors, closing and opening of windows, sending e-mails, providing alerts to human operators, and providing audible alarms.

13. The method for indoor biological detection of a monitored space of claim 1, including the steps of identifying where a source of the plume event started, when the plume detector started, and determining a best location to collect and prepare the air sample.

14. The method for indoor biological detection of a monitored space of claim 1, including the step of providing a video feed from a closed-circuit camera to confirm or disconfirm human activity associated with a biological agent release.

15. The method for indoor biological detection of a monitored space of claim 1, wherein the steps occur in real-time.

16. The method for indoor biological detection of a monitored space of claim 1, wherein some steps occur offline.

17. The method for indoor biological detection of a monitored space of claim 1, wherein the steps

18. An indoor biological detection system for a monitored space, comprising:
(a) a trigger subsystem comprising an array sensors for monitoring air for an aerosol plume, to detect and characterize a plume event;
(b) a presumptive identification subsystem comprising a system to collect and prepare an air sample upon detection of a plume event, and to assay the sample and identify threat organisms; and
(c) a command and control subsystem to initiate a precautionary response, locate a source of the plume, map plume movement within the monitored space, and initiate a protective response.

19. The indoor biological detection system of claim 18, wherein the command and control subsystem archives data regarding the air sample.

20. The indoor biological detection system of claim 18, wherein the presumptive identification subsystem comprises an air collector, a sample preparation and delivery accessory, and a field screening device.

21. The indoor biological detection system of claim 18, wherein the trigger subsystem comprises commercial-off-the-shelf sensors.

22. The indoor biological detection system of claim 18, wherein the trigger subsystem comprises a second tier of sensors comprising an array of bioaerosol sensors that detect biological organisms in the aerosol plume.

23. The indoor biological detection system of claim 20, wherein the field screening device is a Polymerase Chain Reaction (PCR) analysis device.
